(19) United States
(12) Patent Application Publication
(10) Pub. No.: US 2026/0263236 A1
(43) Pub. Date: Sep. 10, 2026

(54) IMPLANT APPLICATOR DEVICE

(71) Applicant: MERIL HEALTHCARE PVT. LTD, Vapi (IN)

(72) Inventors: DARSHAN A. LAD, Navsari (IN); JIMESH B. GANDHI, Bilimora (IN); PRATIK M. PANCHAL, Vapi (IN); LATHEESH KV, Kannur (IN)

(73) Assignee: MERIL HEALTHCARE PVT LTD, Vapi, GJ (IN)

(21) Appl. No.: 19/491,337

(22) PCT Filed: Jun. 7, 2024
(86) PCT No.: PCT/IN2024/050700

§ 371 Date: Dec. 8, 2025

(30) Foreign Application Priority Data
Jun. 7, 2023 (IN) 202321039103

(51) Int. Cl. A61F 2/46

(52) U.S. Cl. CPC A61F 2/4607; A61F 2002/4627; A61F 2002/4629

(57) ABSTRACT

The present invention discloses a device including an adapter, a guide member and a connector. The adapter includes a lumen extending between a proximal end and a distal end of the adapter. A tubular portion of the adapter includes one or more projections disposed towards the distal end of the adapter. A flange portion of the adapter is disposed coaxially to the tubular portion at the proximal end of the adapter. At least one set of a plurality of cavities are disposed circumferentially around the flange portion. The guide member is removably coupled to at least one of the cavities of the flange portion of the adapter to set an anteversion angle. A head portion of the connector is disposed at a proximal end of the connector. A shank portion of the connector is at least partially disposed within the lumen of the adapter through the flange portion.

### FIELD OF INVENTION

The present disclosure relates to a medical device applicator. More particularly, the present disclosure relates to a bone implant applicator.

### BACKGROUND OF INVENTION

Advancing age, injuries, diseases can weaken the bones, joints and/or ligaments of the human body. Such injuries, damages usually appear in the form of physical degeneration which limits the freedom of mobility of the affected person.

In order to correct these damages, additional support or prosthesis may be inserted by way of surgery to supplement the strength of the diseased bone and/or joint. Usually, a mallet is used to hammer the prosthesis within a bone cavity of the affected bone. Thereafter, the prosthesis may be coupled with other components to form an artificial joint, if required, to restore the freedom of mobility of the affected person.

However, the above-described procedure carried out by conventional instruments fail to provide a means to accurately align the prosthesis during impaction of the implant within the bone cavity. More often than not, the prosthesis is prone to slip, subside and/or rotate during the impaction of the prosthesis. Further, due to the use of a mallet, the impaction force used to drive the prosthesis in the bone cavity varies from user to user. Therefore, the underlying bone is susceptible to high risk of fractures due to application of uncontrolled forces.

Thus, there arises a need for a device that overcomes the problems associated with the conventional devices.

### SUMMARY OF INVENTION

Particular embodiments of the present disclosure are described herein below with reference to the accompanying drawings, however, it is to be understood that the disclosed embodiments are mere examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The present disclosure relates to a device including an adapter, a guide member and a connector. The adapter includes a proximal end, a distal end and a lumen extending between the proximal end and the distal end. A tubular portion of the adapter includes one or more projections disposed towards the distal end of the adapter. The projections removably couple the tubular portion to an implant. The tubular portion at least partially define the lumen of the adapter. A flange portion of the adapter is disposed coaxially to the tubular portion at the proximal end of the adapter. The flange portion at least partially defining the lumen of the adapter. At least one set of a plurality of cavities are disposed circumferentially around the flange portion. Each cavity being at an offset with respect to the other cavity. Each cavity of the plurality of cavities corresponds to an anteversion angle being made by the implant coupled to the tubular portion of the adapter with an implantation site. The guide member is removably coupled to at least one of the cavities of the flange portion of the adapter to set the anteversion angle. The connector includes a proximal end and a distal end. A head portion of the connector is disposed at a proximal end of the connector. A shank portion of the connector is at least partially disposed within the lumen of the adapter through the flange portion. The distal end of the shank portion is operationally coupled to the implant for insertion within a bone cavity.

### BRIEF DESCRIPTION OF ACCOMPANYING DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentality disclosed herein. Moreover, those in the art will understand that the drawings are not to scale.

FIG. 1 depicts a device 100 according to an embodiment of the present disclosure.

FIG. 2 depicts an adapter 110, a connector 120 and a guide member 130 of the device 100 according to an embodiment of the present disclosure.

FIG. 2a depicts a cross-sectional view of the adapter 110 of the device 100 according to an embodiment of the present disclosure.

FIG. 2b depicts the adapter 110 of the device 100 coupled to an implant 200 according to an embodiment of the present disclosure.

FIG. 2b1 depicts a longitudinal cross-section of the implant 200 according to an embodiment of the present invention.

FIGS. 2c and 2e depicts the adapter 110 to be used for anterior surgical approach according to an embodiment of the present disclosure.

FIGS. 2d and 2f depicts the adapter 110 to be used for posterior surgical approach according to an embodiment of the present disclosure.

FIG. 3 depicts a cross-sectional view of the adapter 110 of the device 100 coupled to the connector 120 according to an embodiment of the present disclosure.

FIG. 3a depicts a cross-sectional view of the adapter 110 of the device 100 coupled to the connector 120 according to another embodiment of the present disclosure.

FIG. 3a1 depicts the connector 120 according to another embodiment of the present disclosure.

FIGS. 4 and 4a depicts the device 100 at an anteversion angle of 0 degrees according to an embodiment of the present disclosure.

FIGS. 5 and 5a depicts the device 100 at an anteversion angle of 20 degrees according to an embodiment of the present disclosure.

FIGS. 6 and 6a depicts an impactor 160 of the device 100 having a first sub unit 160a and a second sub unit 160b according to an embodiment of the present disclosure.

FIGS. 7 and 7a depicts the impactor 160 of the device 100 having the first sub unit 160a and the second sub unit 160b according to an embodiment of the present disclosure.

FIG. 8 illustrate a method 300 to assemble the device 100 according to an embodiment of the present disclosure.

FIG. 9 illustrate an implantation procedure 400 to insert the implant 200 at an implantation site according to an embodiment of the present disclosure.

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like. Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

The present disclosure relates to an implant applicator device (or device). The device of the present disclosure is used to align (or orient) and insert an implant within a bone cavity (or implantation site). For example, the device of the present disclosure is used to insert a stem prosthesis within a cavity of the femur bone.

Although the present disclosure is described with examples of a stem prosthesis, the teachings of the present disclosure is equally applicable to other osteo-implants and the same is within the scope of the teachings of the present disclosure.

The device of the present disclosure includes an adapter removably coupled to an implant. The adapter is provided with one or more indicators that enable the user to correctly orient and couple the implant to the adapter of the device. The said indicator indicates at least one of a medial side and a lateral side of the implant with respect to the adapter.

At least two different variants of the adapter are made available to a surgeon, where one variant is used for an anterior surgical approach and the other variant is used for posterior surgical approach. In other words, the adapters are provided with distinctive medial notation(s) and/or lateral notation(s) to enable the surgeon to select the adapter with respect to the surgical approach determined to insert the implant within the bone cavity. The surgical approach and the corresponding adapter to be used is determined by the surgeon basis circumstantial facts of the patient.

The adapter is further provided with at least one projection. The projection(s) help to maintain the orientation of the implant with respect to the adapter during the implantation procedure. In other words, the projections prevent the implant from rotating with respect to the adapter during the implantation procedure.

The adapter also helps to set a rotational (or angular) orientation of the implant with respect to the implantation site during implantation. The adapter of the device includes a plurality of cavities that at least partially extends circumferentially around the adapter. Each of the plurality of cavities of the adapter corresponds to a pre-determined anteversion angle. At least a few of the plurality of cavities are disposed diametrically opposite to each other (for example, as a mirror image of each other), thereby enabling the surgeon to use the device on either of the limb (i.e., left limb or right limb) as required.

The device further includes a guide member and an impactor. The guide member helps to align the adapter of the device as well as the implant with respect to the implantation site by selectively orienting and coupling the guide member to one of the plurality of cavities of the adapter, thereby setting the anteversion angle as required. During the implantation procedure, the guide member is aligned with at least one anatomical landmark feature of the implantation site (for example, long axis of the tibia bone), thereby aligning the adapter and the implant with the implantation site.

The impactor enables the user to apply a constant force to drive the implant within the implantation site, irrespective of the user using the device.

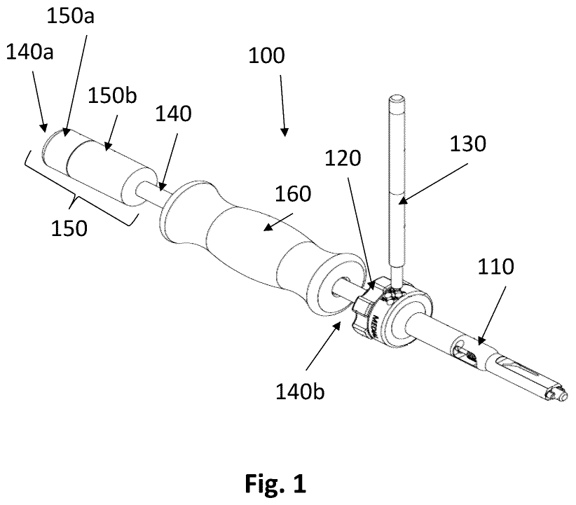

Now referring to the figures, FIG. 1 illustrates an exemplary embodiment of a device 100. The device 100 may include a plurality of components operationally coupled to each other. In an exemplary embodiment, as shown in FIG. 1, the device 100 includes an adapter 110, a connector 120, a guide member 130, an elongate member 140, one or more stoppers 150, and at least one impactor 160.

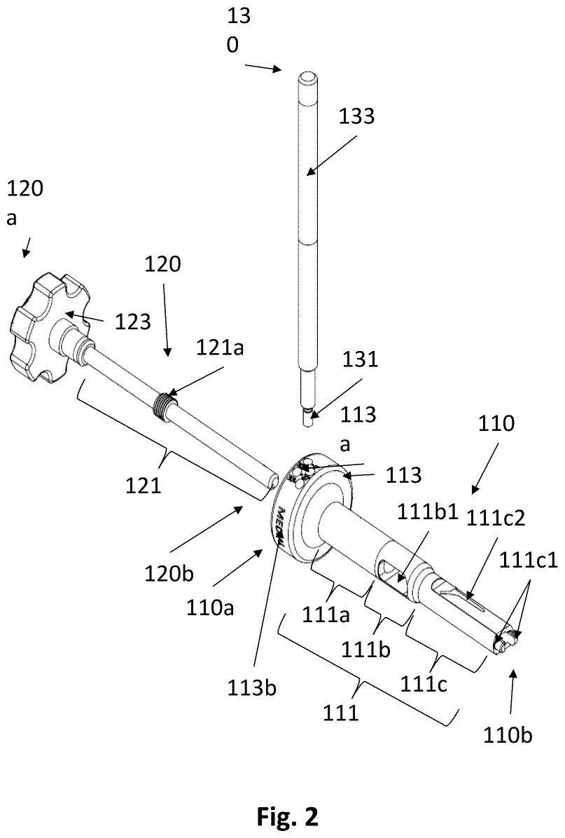

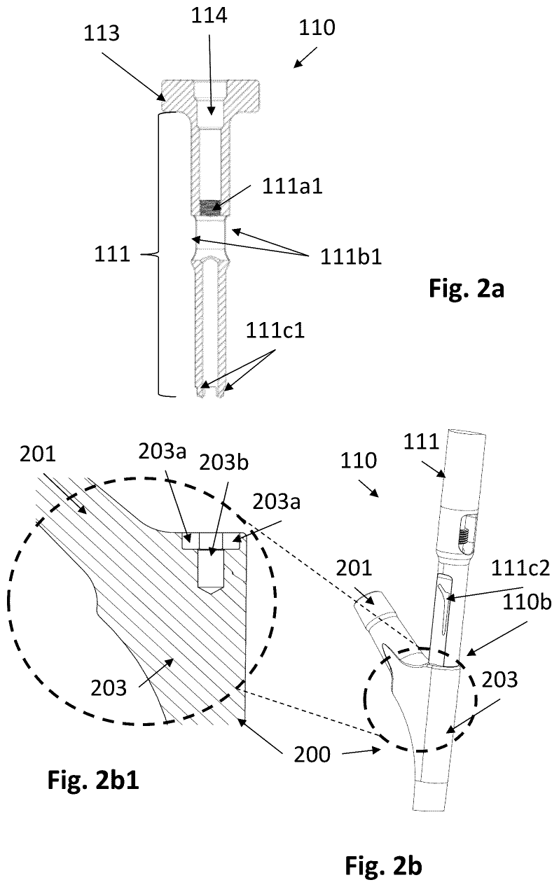

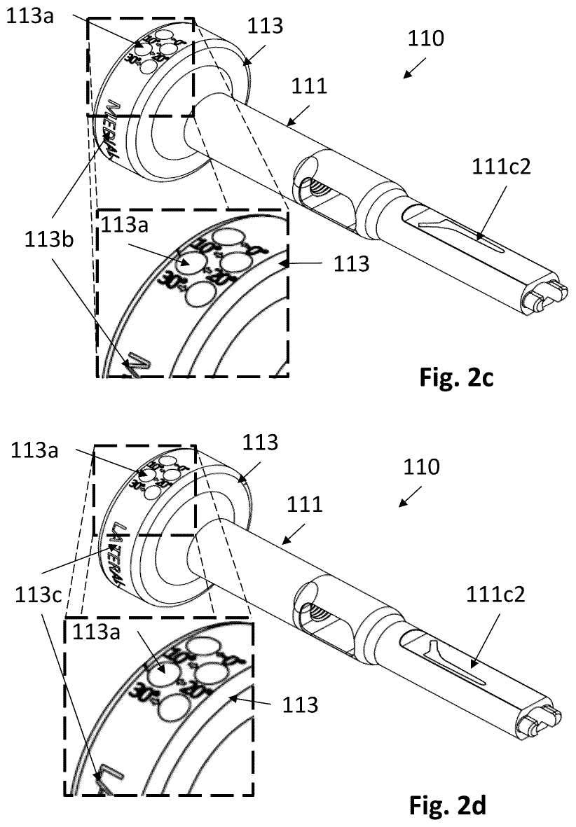

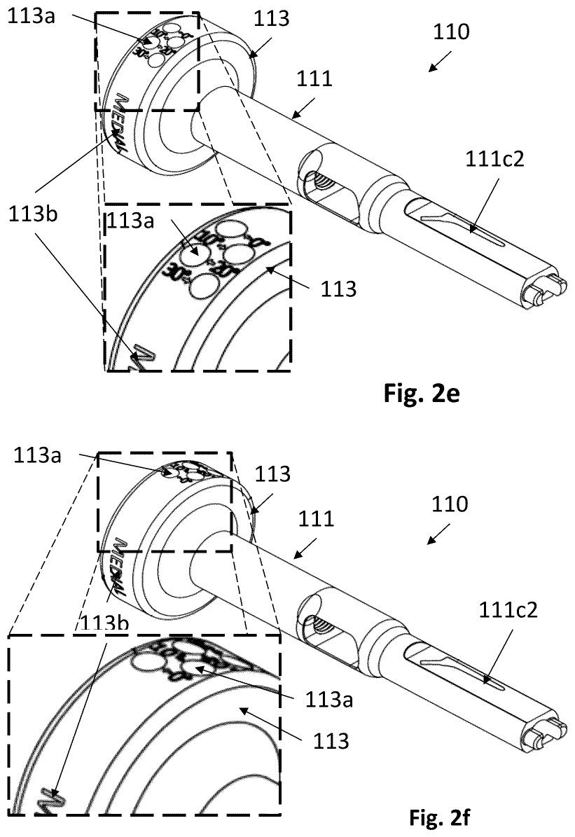

FIG. 2 depicts the adapter 110, the connector 120 and the guide member 130 of the device 100, according to an embodiment of the present disclosure. The adapter 110 of the device 100 includes a proximal end 110a and a distal end 110b. The adapter 110 may be made of a metal or metallic alloys including, but not limited to, Cobalt chromium, titanium, stainless steel, etc. Alternatively, the adapter 110 may be made of a polymeric material including, but not limited to, Polyphenylsulfone (PPSU), Acetal Polyoxymethylene Copolymer (POM-C), carbon fiber, reinforced plastic, etc. In an exemplary embodiment, the adapter 110 is made of biocompatible stainless steel.

The adapter 110 may include a tubular portion 111 disposed at the distal end 110b of the adapter 110 and a flange portion 113 disposed at the proximal end 110a of the adapter 110. The tubular portion 111 and the flange portion 113 may be removably coupled to each other via, without limitation, mechanical fasteners, metal/plastic/composite/hybrid material joining methods like welding, press-fit, threading, etc. Alternatively, the tubular portion 111 and the flange portion 113 forms an integral structure.

The tubular portion 111 and the flange portion 113 may together at least partially form (or define) a lumen 114 (as shown in FIG. 2a), such that the lumen 114 extends from the proximal end 110a and the distal end 110b of the adapter 110. The lumen 114 of the adapter 110 may be configured to at least partially receive the connector 120 (described below). In an exemplary embodiment, the adapter 110 includes a circular shaped lumen 114.

The tubular portion 111 may have a length of at least 10 mm. The tubular portion 111 may have a diameter of at least 2 mm. In an exemplary embodiment, the length and diameter of the tubular portion 111 are 107 mm and 15 mm, respectively. The tubular portion 111 may have any shape including, but not limited to, cylindrical, elliptical, etc.

The tubular portion 111 includes one or more sections. In an exemplary embodiment, as shown in FIG. 2, the tubular portion 111 includes three sections, namely a first section 111a, a second section 111b and a third section 111c. The first section 111a is disposed towards the proximal end 110a and adjacent to the flange portion 113. In an exemplary embodiment, as show in FIG. 2, the first section 111a is cylindrical in shape which enables the user to handle and interact with the adapter 110.

The first section 111a, towards the proximal end 110a, includes a first engagement portion 111a1 extending at least partially across a length of the first section 111a. The first engagement portion 111a1 transiently engages the connector 120 when the connector 120 is inserted within the lumen 114 of the adapter 110 (described below). The first engagement portion 111a1 prevents unintentional withdrawal/retraction of the connector 120 during the implantation procedure. In an exemplary embodiment, as shown in FIG. 2a, the first engagement portion 111a1 includes a plurality of female threads on an inner surface of the first section 111a, i.e., within the lumen 114 defined by the first section 111a of the tubular portion 111.

The first engagement portion 111a1 further helps the surgeon to selectively withdraw the connector 120 from within the lumen 114 of the adapter 110 (described below) to clean/sterilize/repair the device 100 after/before the implantation procedure.

The second section 111b is disposed between the first section 111a and the third section 111c. Because of the second section 111b, the connector 120 is able to freely rotate within the lumen 114 of the adapter 110 thus, enabling the surgeon to couple an implant 200 (as shown in FIG. 2b) with the connector 120.

Additionally or optionally, as shown in FIG. 2, the second section 111b of the tubular portion 111 is provided with one or more cut outs 111b1, thereby at least partially revealing the lumen 114 thereunder. The cut outs 111b1 may have any pre-defined shape and/or angular orientation. In an exemplary embodiment, as shown in FIGS. 2 and 2a, the second section 111b includes two diametrically opposite thorough rounded rectangular cut outs 111b1, that is perpendicular to the lumen 114 of the adapter 110. The cut outs 111b1 enable the connector 120 to freely rotate within the lumen 114 of the adapter 110 without letting the connector 120 slip out of the lumen 114 of the adapter 110 (described below).

The third section 111c is disposed towards the distal end 110b and adjacent to the second section 111b. The third section 111c, at the distal end 110b, is provided with one or more projections 111c1. The projections 111c1 are coupled to the third section 111c of the adapter 110 via, without limitation, mechanical fasteners, metal/plastic/composite/hybrid material joining methods like welding, press-fit, threading, etc. In an exemplary embodiment, as shown in FIG. 2, two projections 111c1 are integrally formed with the tubular portion 111 at the distal end 110b of the adapter 110. The projections 111c1 restrict rotation of the implant 200 with respect to the adapter 110, thereby maintaining a pre-defined orientation of the implant 200 during the implantation procedure.

FIG. 2b depicts an exemplary embodiment of the implant 200 coupled to the adaptor 110. The implant 200 is made of a material including but not limited to stainless steel, titanium, cobalt chromium, etc. The implant 200 may be configured to be implanted within a bone cavity (not shown), for example, inside a reamed cavity of a femur. The implant 200 may include a neck portion 201 and a stem portion 203. The neck portion 201 may define a pre-defined angle with the stem portion 203 corresponding to a femoral neck angle of the patient. In an embodiment, the pre-defined angle ranges from 110° to 150°.

In an exemplary embodiment, as shown in FIG. 2b, the stem portion 203 of the implant 200 is removably coupled with the tubular portion 111 of the adapter 110. As shown in FIG. 2b1, the stem portion 203 of the implant 200 is provided with one or more first recesses 203a corresponding to each of the projections 111c1 of the adapter 110. Thus, when the implant 200 is coupled to the adapter 110, the projections 111c1 of the adapter 110 engage with corresponding first recesses 203a of the stem portion 203. The said engagement between the projections 111c1 of the adapter 110 and the first recesses 203a of the implant 200 restricts rotation of the implant 200 with respect to the adapter 110, thereby maintaining a pre-defined orientation of the implant 200 during the implantation procedure.

Additionally or optionally, to correctly orient the implant 200 with the adapter 110, the tubular portion 111 may be provided with at least one first indicator 111c2.

In an exemplary embodiment, as shown in FIGS. 2 and 2b, the first indicator 111c2 is provided over the third section 111c of the tubular portion 111. The first indicator 111c2 is an illustration of the implant 200 indicating a position of the implant 200 and/or a direction of the neck portion 201 of the implant 200 with respect to the adapter 110.

In an embodiment, a second indicator (not shown), similar to the first indicator 111c2, is provided on the tubular portion 111 of the adapter 110. In an exemplary embodiment, the second indicator is disposed diametrically opposite to the first indicator 111c2. The first indicator 111c2 and the second indicator enable the surgeon to use the same adapter 110 for inserting the implant 200 in any of the limbs (i.e., right limb or the left limb) of the patient as required. For example, the surgeon may refer the first indicator 111c2 when inserting the implant 200 within the femur bone of the right limb and refer the second indicator when inserting the implant 200 within the femur bone of the left limb.

In an embodiment, the tubular portion 111 is provided with one first indicator 111c2. In an alternate embodiment, the tubular portion 111 is provided with one second indicator. In yet another alternate embodiment, the tubular portion 111 is provided with one first indicator 111c2 and one second indicator.

Although the tubular portion 111 is described as having a cylindrical-like structure, other functionally equivalent shape of the tubular portion 111 is within the scope of the teachings of the present disclosure. For example, the tubular portion 111 may include a stepped bar-like shape.

Referring back to FIG. 2, the flange portion 113 of the adapter 110 may be coaxially disposed around the tubular portion 111, towards the proximal end 110a. The flange portion 113 may have a length of at least 5 mm. The flange portion 113 may have a diameter of at least 10 mm. In an exemplary embodiment, the length and diameter of the flange portion 113 are 12.6 mm and 37.8 mm respectively.

In an exemplary embodiment, as shown in FIG. 2, the flange portion 113 is cylindrically shaped. Although the flange portion 113 is depicted as cylindrically shaped, other functionally equivalent shapes of the flange portion 113 are within the scope of the teachings of the present disclosure.

The flange portion 113 may be provided with at least one set of a plurality of cavities 113a configured to receive the guide member 130 (described below). The cavities 113a may be disposed circumferentially offset from each other such that each cavity 113a corresponds to a pre-defined anteversion angle (described below). The anteversion angle may range from −50° to 80°.

Although the adapter 110 is described with examples of cavities 113a disposed on the flange portion 113, the cavities 113a may instead be disposed on the tubular portion 111 and the same is within the scope of the present disclosure.

The anteversion angle of the cavities 113a may extend (or increase) in a pre-defined direction, i.e., either clockwise direction or anticlockwise direction (described below). Alternatively, the anteversion angle of the cavities 113a may extend (or increase) either towards a medial side or towards a lateral side of the adapter 110 (described below). The medial plane corresponds to the direction of the neck portion 201 of the implant 200.

In an exemplary embodiment, as shown in FIG. 2, flange portion 113 is provided with one set of four cavities 113a. The cavities 113a are circumferentially offset from each other by 10 degrees according to an embodiment. Thus, the cavities 113a correspond to the anteversion angle ranging from 0° to 30°, i.e., respective cavities 113 a correspond to 0°, 10°, 20°, and 30° in the clockwise direction when viewed from the proximal end 110a of the adapter 110).

In yet another exemplary embodiment, not shown, the flange portion 113 is provided with two sets of four cavities 113a each, namely a first set of cavities 113a and a second set (not shown) of cavities 113a. The first set of cavities 113a is disposed circumferentially around the flange portion 113 as depicted in FIG. 2. The second set of cavities 113a is disposed diametrically opposite to the first set of four cavities 113a. Therefore, the flange portion 113 is provided with a total of eight cavities 113a. The first set of cavities 113a and the second set of cavities 113a enable the surgeon to use the same adapter 110 for inserting and aligning the implant 200 with any of the limbs (i.e., right limb or the left limb) of the patient as required.

According to an embodiment, the flange portion 113 is provided with at least one of a third indicator(s) 113b (as shown in FIGS. 2 and 2c) and a fourth indicator(s) 113c (as shown in FIG. 2d). The third indicator 113b and the fourth indicator 113c are disposed diametrically opposite to each other. A pre-defined orientation of the third indicator 113b and/or the fourth indicator 113c with respect to the anteversion angle(s) (described below) enables the surgeon to determine and align the medial side and the lateral side of the implant 200 with a medial side and a lateral side of the adapter 110 during the implantation procedure.

In an embodiment, the flange portion 113 is provided with one third indicator 113b. In an alternate embodiment, the flange portion 113 is provided with one fourth indicator 113c. In yet another alternate embodiment, the flange portion 113 is provided with one third indicator 113b and one fourth indicator 113c.

In an exemplary embodiment, as shown in FIGS. 2 and 2c, the third indicator 113b includes a “MEDIAL” notation marked over the flange portion 113, thus depicting the medial side of the adapter 110. In an exemplary embodiment, as shown in FIG. 2d, the fourth indicator 113c includes a “LATERAL” notation marked over the flange portion 113, thus depicting the lateral side of the adapter 110. The orientation of the medial notation(s) and/or lateral notation(s) with respect to the anteversion angle(s) enable the surgeon to select the adapter 110 with respect to the anterior surgical approach or the posterior surgical approach to insert the implant 200 within the bone cavity (described below).

The directional property associated with the anteversion angle of the cavities 113a along with the “MEDIAL” and “LATERAL” notations help determine the surgical approach in which the adapter 110 is to be used by the surgeon. For example, FIGS. 2c and 2d depict two different orientations of the adapter 110. The “MEDIAL” and “LATERAL” notations of the adapters 110 depicted in FIGS. 2c and 2d are swapped with each other. Accordingly, the first indicator 111c2 (and the second indicator) illustrating the direction of the neck portion 201 of the implant 200 is also reversed. Thus, the adapter 110 illustrated in FIG. 2c depicts the increase in anteversion angle of the cavities 113a from the “LATERAL” notation towards the “MEDIAL” notation. Conversely, the adapter 110 illustrated in FIG. 2d depicts the increase in anteversion angle of the cavities 113a from the “MEDIAL” notation towards the “LATERAL” notation. Accordingly, the adapter 110 depicted in FIG. 2c is used by the surgeon for anterior surgical approach and the adapter 110 depicted in FIG. 2d is used by the surgeon for posterior surgical approach.

In yet another example, depicted in FIGS. 2e and 2f, the adapters 110 have their respective anteversion angle increase in opposite direction (instead of swapping the “MEDIAL” (third indicator 113b) and “LATERAL” (fourth indicator 113c) notations of the adapter 110 and the direction of the first indicator 111c2 as described above). Thus, the adapter 110 illustrated in FIG. 2e depicts the decrease in anteversion angle of the cavities 113a from the “MEDIAL” notation towards the “LATERAL” notation. Conversely, the adapter 110 illustrated in FIG. 2f depicts the increase in anteversion angle of the cavities 113a from the “MEDIAL” notation towards the “LATERAL” notation. Accordingly, the adapter 110 depicted in FIG. 2e is used by the surgeon for anterior surgical approach and the adapter 110 depicted in FIG. 2f is used by the surgeon for posterior surgical approach.

Although the orientation of the anteversion angles is described with both the “MEDIAL” notation as well as the “LATERAL” notation, only one of the said notations may be enough to select the adapter 110 either for anterior or posterior surgical approach as determined by the surgeon. Accordingly, either one of the notations or both the notations may be provided over the flange portion 113 of the adapter 110.

Referring back to FIG. 2, an embodiment of the connector 120 is depicted. The connector 120 may be made of a metal or metallic alloys including but not limited to any surgical steels, alloys including Cobalt chromium, titanium, stainless steel, etc. Alternatively, the connector 120 may be made of a polymeric material including, but not limited to, Polyphenylsulfone (PPSU), Acetal Polyoxymethylene Copolymer (POM-C), carbon fiber, reinforced plastic. In an exemplary embodiment, the connector 120 is made of biocompatible stainless steel. The connector 120 may include a shank portion 121 and a head portion 123. The connector 120 includes a proximal end 120a and a distal end 120b. The head portion 123 may be disposed at the proximal end 120a and the shank portion 121 may be disposed at the distal end 120b. The shank portion 121 and the head portion 123 may be removably or fixedly coupled to each other. Alternatively, the shank portion 121 forms an integral structure with the head portion 123.

The shank portion 121 may have a shape corresponding to the shape of the lumen 114 of the adapter 110. The shank portion 121 may have a length and a diameter depending upon the length and diameter of the lumen 114 of the adapter 110. In an exemplary embodiment, the length and diameter of the shank portion 121 are 111 mm and 6 mm respectively.

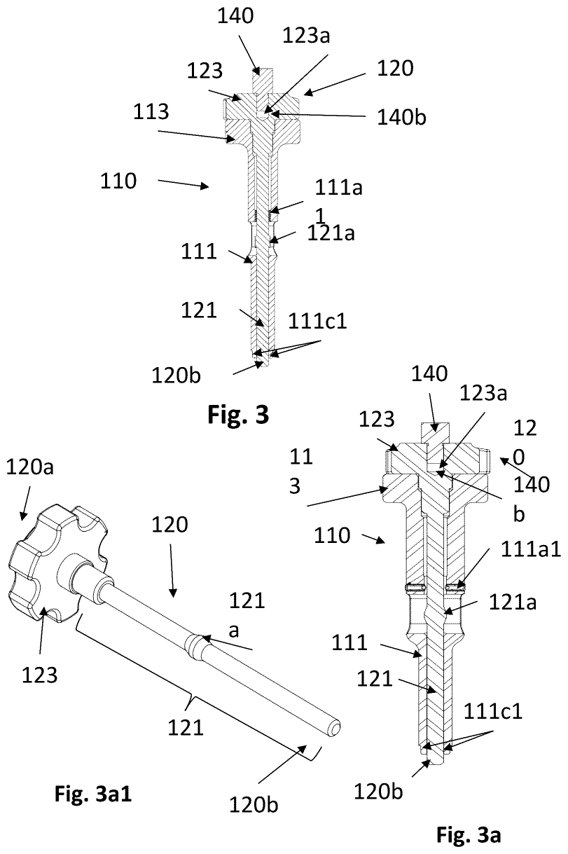

The shank portion 121 of the connector 120 is at least partially disposed within the lumen 114 of the adapter 110 through the flange portion (113). In an exemplary embodiment, as shown in FIG. 3, the shank portion 121 of the connector 120 is disposed within the lumen 114 of the adapter 110 such that the distal end 120b of the connector 120 emerges out of the distal end 110b of the adapter 110.

The distal end 120b of the connector 120 (and/or the shank portion 121) may be operationally coupled to the implant 200. As shown in FIG. 2b1, the stem portion 203 of the implant 200 is provided with a second recess 203b configured to receive the distal end 120b of the connector 120. In an exemplary embodiment, not shown, the distal end 120b of the connector 120 and the second recess 203b of the stem portion 203 of the implant 200 are each provided with corresponding threads such that the distal end 120b of the connector 120 is fastened into the second recess 203b of the stem portion 203. The surgeon may screw/unscrew the connector 120 to the implant 200 to couple/uncouple the connector 120 and the implant 200. Other functionally equivalent mechanical means of coupling the connector 120 and the implant 200 are within the scope of the teachings of the present disclosure.

As shown in FIG. 2, the shank portion 121 is provided with a second engagement portion 121a extending at least partially across a length of the shank portion 121. The second engagement portion 121a is configured to transiently engage the first engagement portion 111a1 of the adapter 110. In an exemplary embodiment, the second engagement portion 121a includes a plurality of male threads corresponding to the female threads of the first engagement portion 111a1 of the adapter 110.

The shank portion 121 of the connector 120 is at least partially inserted within the lumen 114 of the adapter 110 by sliding the shank portion 121 through the flange portion 113. The connector 120 may be toggled between a first configuration and a second configuration depending upon a position of the second engagement portion 121a with respect to the first engagement portion 111a1 when the shank portion 121 is at least partially inserted within the lumen 114. In the first configuration of the connector 120, the second engagement portion 121a is disposed proximal to the first engagement portion 111a1, i.e., the second engagement portion 121a is disposed within the first section 111a of the adapter 110. In the first configuration of the connector 120, the connector 120 is slidably retractable from the adapter 110 to, for example, clean and sterilize the device 100.

In the second configuration of the connector 120, as shown in FIG. 3, the second engagement portion 121a is disposed distal to the first engagement portion 111a1, i.e., the second engagement portion 121a is disposed within the second section 111b of the adapter 110. In the second configuration of the connector 120, the connector 120 is free to rotate within the lumen 114 of the adapter 110 such that the surgeon may rotate the connector 120 to couple the implant 200 with the connector 120 before commencing the implantation procedure. In the second configuration of the connector 120, the connector 120 is restricted from being slidably retractable from the adapter 110 as the first engagement portion 111a1 of the adapter 110 hinders the second engagement portion 121a of the connector 120 thereby preventing accidental removal of the connector 120 from within the adapter 110 during the implantation procedure.

To toggle the connector 120 between its first configuration and second configuration, the second engagement portion 121a is first physically engaged with the first engagement portion 111a1 and a torque is applied to rotate the second engagement portion 121a with respect to the first engagement portion 111a1. Thus, the said transient engagement between the first engagement portion 111a1 and the second engagement portion 121a toggles the connector 120 between its first configuration and second configuration. In an exemplary embodiment, the torque is applied to the connector 120 in a clockwise direction to toggle the connector 120 from its first configuration to its second configuration and vice versa.

In an alternate exemplary embodiment, as shown in FIGS. 3a and 3a1, the first engagement portion 111a1 includes at least one plunger (or other functionally equivalent means) and the second engagement portion 121a includes a bulged portion (or other functionally equivalent means. In this embodiment, the bulged portion of the connector 120 is manually pulled/pushed across the plunger to toggle the connector 120 between its first configuration and second configuration.

Although coupling between the first engagement portion 111a1 and the second engagement portion 121a is described with the examples of threads, bulged-plunger portion, other functionally equivalent means are within the scope of the teachings of the present disclosure.

The head portion 123 of the connector 120 helps the surgeon to interact with the connector 120, for example, enables the surgeon to apply the torque to the connector 120. Thus, the head portion 123 of the connector 120 enables the surgeon to rotate the connector 120 to either toggle the connector 120 between its first configuration and second configuration and couple the connector 120 to the implant 200. Accordingly, the head portion 123 of the connector 120 may be ergonomically shaped to enable the user to comfortably grasp and manipulate (for example, rotate) the head portion 123. In an exemplary embodiment, depicted in FIG. 2, the head portion 123 of the connector 120 is shaped like a roller with grooves.

As shown in FIGS. 3 and 3a, the head portion 123 is provided with at least one cavity 123a. The cavity 123a is configured to receive the elongate member 140 of the device 100 (described below).

The guide member 130 may have a shape including but not limited to cylindrical, spherical, hexagonal, elliptical, triangular, etc. In an exemplary embodiment, as shown in FIG. 2, the guide member 130 is rod shaped. The guide member 130 may be used to align the device 100 as well as the implant 200 with the implantation site with reference to the patient's anatomy (described later).

The guide member 130 may be provided with at least one axial projection 131. In an exemplary embodiment, the axial projection 131 forms an integral structure with the guide member 130.

The axial projection 131 of the guide member 130 may be removably coupled to at least one of the cavities 113a of the adapter 110. In an exemplary embodiment, the axial projection 131 and the cavities 113a are provided with complimenting threads such that the axial projection 131 is screwed into one of the cavities 113a of the adapter 110. In an alternative embodiment, the axial projection 131 is snap-fitted into one of the cavities 113a of the adapter 110. Other means of coupling the guide member 130 to the cavities 113a of the adapter 110 are within the scope of the teachings of the present disclosure.

Additionally or optionally, as shown in FIG. 2, the guide member 130 may be provided with a rough surface 133 extending at least partially across a length of the guide member 130. The rough surface 133 enables the surgeon to easily handle and/or hold the guide member 130.

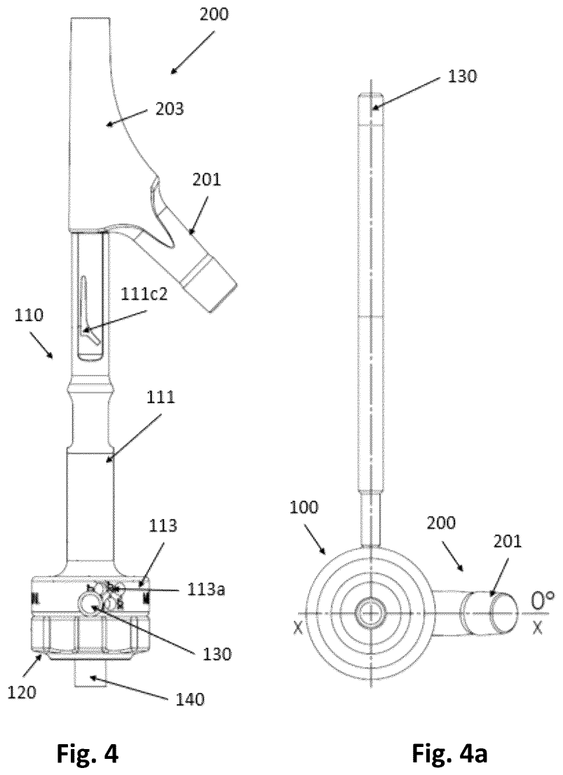

FIG. 4 partially depicts an exemplary embodiment of the device 100 coupled to the implant 200 (as described above). As shown in FIG. 4, the guide member 130 is coupled to one cavity 113a of the adapter 110 that corresponds to the anteversion angle of zero degrees.

FIG. 4a depicts a view of the device 100 (corresponding to the configuration shown in FIG. 4) as viewed by the surgeon according to an embodiment. FIG. 4a, further, depicts an imaginary axis ‘x-x’ that runs parallel to a coronal plane (not shown) of the patient. The surgeon aligns the guide member 130 of the device 100 perpendicular to the imaginary axis ‘x-x’ by rotating the device 100. In other words, the surgeon aligns the guide member 130 parallel to the long axis of the tibia while the tibia bone is flexed and held perpendicular to the femur bone. The said alignment of the guide member 130, as shown in FIGS. 4 and 4a, results in the neck portion 201 of the implant 200 making the anteversion angle of zero degrees with the imaginary axis ‘x-x’.

The anteversion angle (or Femoral anteversion angle) plays a very crucial role in the success of the implantation procedure as the anteversion angle influences a degree of torsional forces experienced by the femur bone. Any deviations in the anteversion angle, determined by the surgeon for each patient, may negatively affect the biomechanics of the hip through alterations in factors such as movement arm lengths and joint loading.

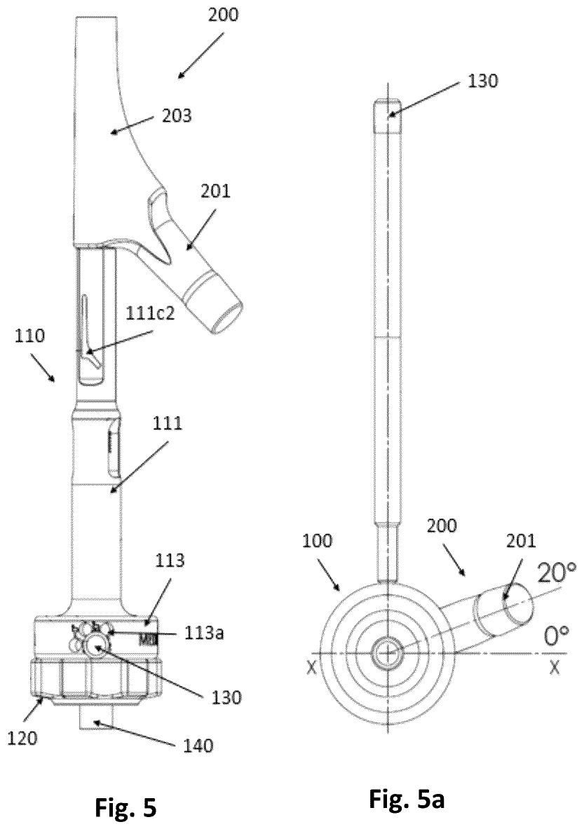

Similar to FIG. 4, FIG. 5 partially depicts an exemplary embodiment of the device 100 coupled to the implant 200 (as described above). As shown in FIG. 5, the guide member 130 is coupled to one cavity 113a of the adapter 110 that corresponds to the anteversion angle of twenty degrees.

FIG. 5a depicts a view of the device 100 (corresponding to the configuration shown in FIG. 5) as viewed by the surgeon, according to one embodiment. The surgeon aligns the guide member 130 of the device 100 perpendicular to the imaginary axis ‘x-x’ by rotating the device 100. In other words, the surgeon aligns the guide member 130 parallel to the long axis of the tibia while the tibia bone is flexed and held perpendicular to the femur bone. The said alignment of the guide member 130, as shown in FIGS. 5 and 5a, results in the neck portion 201 of the implant 200 making the anteversion angle of twenty degrees with the imaginary axis ‘x-x’.

As shown in exemplary depictions in FIGS. 4, 4a, 5 and 5a, the coupling between the axial projection 131 of the guide member 130 and one of the cavities 113a of the adapter 110 enables the surgeon to set the pre-determined anteversion angle of the implant 200 for each patient as required.

Although the device 100 of the present disclosure is described above with two embodiments of anteversion angles, the device 100 of the present disclosure can be used with any number of anteversion angles and the same is within the scope of the teachings of the present disclosure.

Referring back to FIG. 1, the elongate member 140 includes a proximal end 140a and a distal end 140b. The elongate member 140 may have a pre-defined shape including but not limited to cylindrical, spherical, hexagonal, elliptical, triangular, etc. In an exemplary embodiment, the elongate member 140 is shaped cylindrically. The elongate member 140 may have a length of at least 100 mm. The elongate member 140 may have a diameter of at least 5 mm. In an exemplary embodiment, the length and diameter of the elongate member 140 are 241 mm and 10 mm respectively.

As shown in FIGS. 3 and 3a, the distal end 140b of the elongate member 140 may be removably coupled to the cavity 123a of the head portion 123 of the connector 120 via threading, snap fit, etc. Alternately, the distal end 140b of the elongate member 140 may be fixedly coupled to the cavity 123a of the head portion 123 of the connector 120. Accordingly, the elongate member 140 may be rotated by the surgeon in order to rotate the connector 120.

As shown in FIG. 1, the stoppers 150 may be coupled to the proximal end 140a of the elongate member 140 via press fit, snap fit, welding, etc. The stoppers 150 may have a pre-defined shape including but not limited to cylindrical, spherical, hexagonal, elliptical, triangular, etc. In an exemplary embodiment, as shown in FIG. 1, two cylindrically shaped stoppers 150 are coupled to the proximal end 140a of the elongate member 140, namely, a first stopper 150a and a second stopper 150b.

The first stopper 150a may be disposed proximal to the second stopper 150b. The first stopper 150a may be made of a metal or metallic alloys including but not limited to Cobalt chromium, titanium, stainless steel, etc. In an exemplary embodiment, the first stopper 150a is made of stainless steel. The first stopper 150 a may have a length of at least 1 mm. The first stopper 150a may have a diameter of at least 5 mm. In an exemplary embodiment, the length and diameter of the first stopper 150a are 20 mm and 25.5 mm respectively.

The first stopper 150a enables the surgeon, optionally, to use a mallet or the like to impact the first stopper 150a of the device 100 in order to insert the implant 200 within the bone cavity. In an exemplary embodiment, the force from the mallet is transferred from the first stopper 150a to the head portion 123 of the connector 120 via the elongate member 140.

The second stopper 150b may be made of a material including but not limited to silicon, plastic, any load resistant gels, polymeric material, elastomers, etc. Alternatively, the second stopper 150b may include a resilient member. In an exemplary embodiment, the second stopper 150b is made of medical grade Acetal Co-polymer. The second stopper 150b may have a length of at least 5 mm. The second stopper 150 b may have a diameter of at least 2 mm. In an exemplary embodiment, the length and diameter of the second stopper 150 b are 43.5 mm and 25.5 mm respectively. The second stopper 150b helps to absorb any residual forces originating from the impactor 160 (described below).

As shown in FIG. 1, the impactor 160 may be slidably disposed around the elongate member 140, between the stoppers 150 and the head portion 123 of the connector 120. The impactor 160 may be shaped ergonomically for ease of use. The impactor 160 may be made of a metal or metal alloys including but not limited to Cobalt chromium, titanium, stainless steel, etc. In an exemplary embodiment, as shown in FIG. 1, the impactor 160 is made of bio-compatible stainless steel.

Lower density/quality bones (for example, osteoporotic bones) are prone to higher risk of fracture when subjected to high amount of force. Whereas, bones having high density/quality (for example, normal/sclerotic bones) are more tolerant to fracture when subjected to high amount of force. Thus, depending on the bone density/quality at the implantation site, an appropriate weight of the impactor 160 is selected for inserting the implant 200 within the bone cavity (described later).

In an exemplary embodiment, as shown in FIG. 1, the impactor 160 has an integral structure. In an exemplary embodiment, the impactor 160 weighs 1013 grams which is used for osteoporotic bones. In another exemplary embodiment, the impactor 160 weighs 1457 grams which is used for sclerotic bones.

The impactor 160 is slidably used, by the surgeon, to impact the head portion 123 of the connector 120, thereby transferring a pre-defined amount of force to the connector 120 from the impactor 160. The connector 120 transfers the pre-defined force to the implant 200, thereby enabling the implant 200 to be pushed within the bone cavity at the implantation site.

The pre-defined amount of force is directly proportional to at least the weight of the impactor 160 and/or a distance between the stoppers 150 and the connector 120. The said distance may at least be 80 mm. In an exemplary embodiment, the distance between the stoppers 150 and the connector 120 is 171.5 mm. The impactor 160 enables the user to apply a constant force to the implant 200, irrespective of the user.

The impactor 160 is slid over the elongate member 140, by the surgeon, in a to-and-fro motion between the connector 120 and the stopper 150. The second stopper 150b, thus, absorbs any residual forces of the impactor 160 when the impactor 160 returns from the connector 120 to the second stopper 150b.

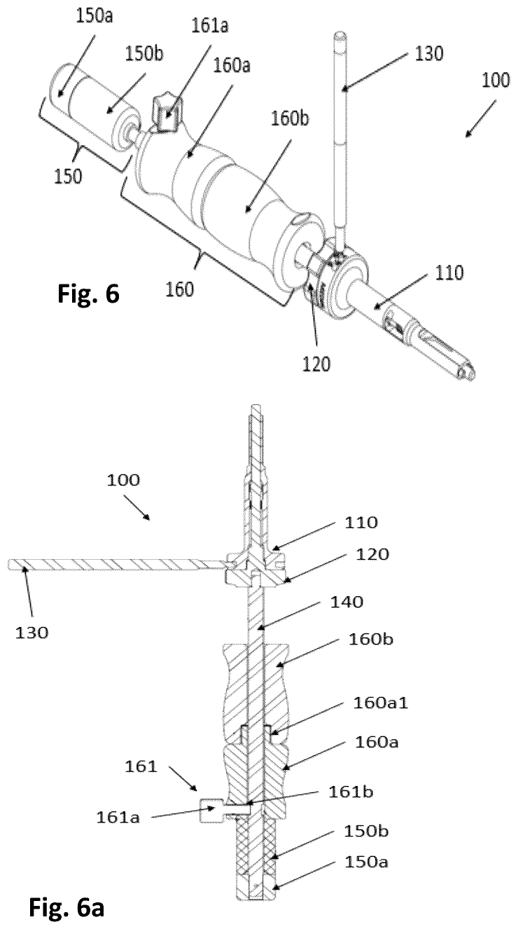

In an alternative embodiment, as shown in FIGS. 6 and 6a, the impactor 160 is made of two or more sub-units, for example, a first sub-unit 160a and a second sub-unit 160b. The first and second sub-units 160a, 160b of the impactor 160 may each have a pre-defined weight of at least 200 grams depending upon user requirements. The first and second sub-units 160a, 160b of the impactor 160 may be selectively assembled over the elongate member 140 to get the required amount of weight (or force) of the impactor 160 as per bone density and/or quality at the implantation site.

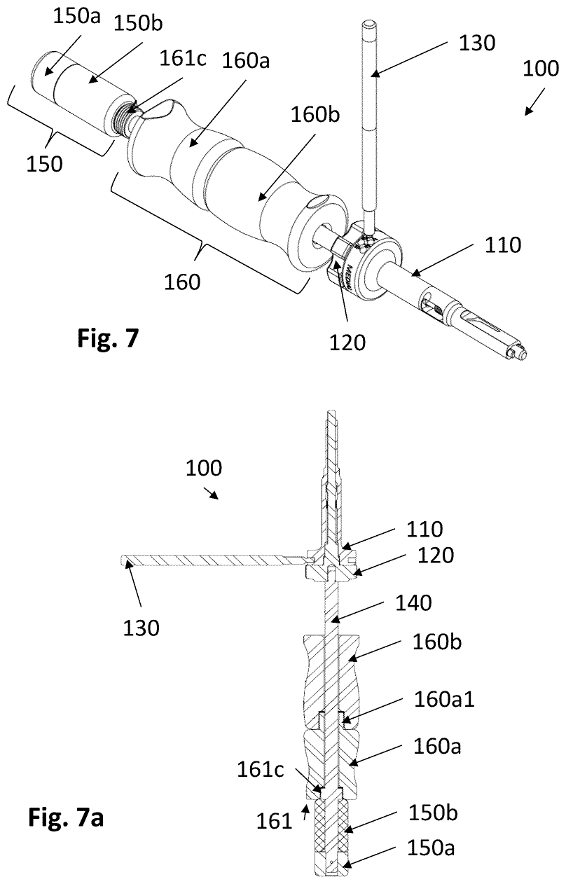

The first sub unit 160a is disposed proximal to the second sub unit 160b. The first sub unit 160a is removably coupled to the second sub unit 160b. In an exemplary embodiment, as shown in FIGS. 6a and 7a, the second sub unit 160b is fastened over a threaded portion 160a1 of the first sub unit 160a. Other functionally equivalent means to couple the first and second sub units 160a, 160b to each other are within the scope of the teachings of the present disclosure.

The impactor 160 may be toggled between a first configuration and a second configuration as required by the surgeon. In the first configuration of the impactor 160, both the first and second sub units 160a, 160b are coupled to each other and configured to slide over the elongate member 140. In the first configuration of the impactor 160, the impactor 160 is used for sclerotic bones.

In the second configuration of the impactor 160, the second sub unit 160b is decoupled from the first sub unit 160a. Further, the first sub unit 160a is reversibly docked adjacent to the second stopper 150b. Thus, only the second sub unit 160b is configured to slide over the elongate member 140. In the second configuration of the impactor 160, the impactor 160 is used for osteoporotic bones.

The first sub-unit 160a may be reversibly docked via a docking means 161 adjacent to the stoppers 150. In an exemplary embodiment, as shown in FIGS. 6 and 6a, the docking means 161 include a knob 161a. The knob 161a is rotatably coupled to the first sub unit 160a via a plurality of male threads. Accordingly, the first sub unit 160a is provided with a hole 161b having a plurality of female threads to at least partially receive the knob 161a.

In an exemplary embodiment, the knob 161a is fastened in the hole 161b of the first sub unit 160a to dock the first sub unit 160a over the elongate member 140. The knob 161a, upon being fastened in the hole 161b, at least partially engages a slot (not shown) of the elongate member 140 thereby preventing the first sub unit 160a from sliding over the elongate member 140. Conversely, the knob 161a is disengaged out of the hole 161b to undock the first sub unit 160a.

FIGS. 7 and 7a, depicts another exemplary embodiment to dock the first sub unit 160a of the impactor 160. In an exemplary embodiment, as shown in FIG. 7, the docking means 161 includes an engagement portion 161c disposed distal to the second stopper 150b over the elongate member 140. The first sub unit 160a may be removably coupled with the engagement portion 161c.

In an exemplary embodiment, the first sub unit 160a and the engagement portion 161c are provided with a plurality of complementing threads to enable the first sub unit 160a to dock over the elongate member 140. Accordingly, as shown in FIG. 7a, the first sub unit 160a is fastened over the engagement portion 161c to prevent the first sub unit 160a from sliding over the elongate member 140.

Other functionally equivalent means to dock the first sub unit 160a over the elongate member 140 are within the scope of the teachings of the present disclosure.

Although the impactor 160 of the present disclosure is described with examples of two sub units, the impactor 160 with more than two sub units are within the scope of the teachings of the present disclosure.

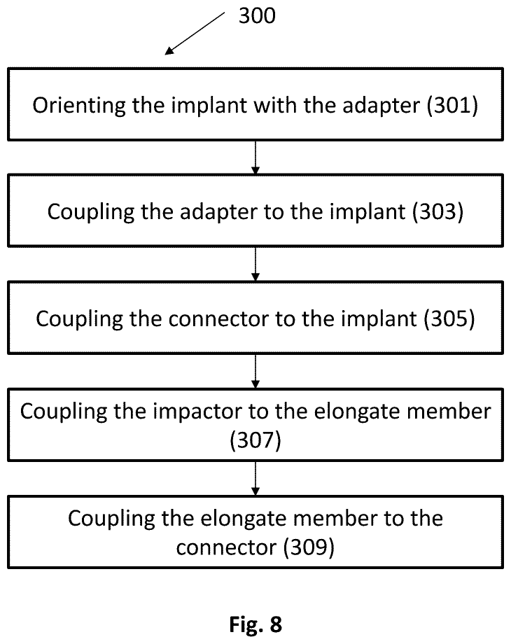

FIG. 8 depicts an exemplary embodiment of a method 300 for assembling the device 100 before commencing the implantation procedure.

The method commences at step 301 by orienting the neck portion 201 of the implant 200 towards the medial side of the adapter 110. The surgeon ensures the orientation of the neck portion 201 of the implant 200 basis at least one of the first indicator 111c2 (and/or the second indicator) provided over the third section 111c of the tubular portion 111, and the third indicator 113b (and/or the fourth indicator 113c) provided over the flange portion 113.

At step 303, the adapter 110 is coupled to the implant 200. In an exemplary embodiment, as shown in FIG. 2b, the stem portion 203 of the implant 200 is removably coupled with the adapter 110. The projections 111c1 of the adapter 110 engage with the stem portion 203 such that rotation of the implant 200 is restricted with respect to the adapter 110, thereby maintaining a pre-defined orientation of the implant 200 during the implantation procedure.

At step 305, the connector 120 is coupled to the implant 200. In an exemplary embodiment, not shown, the distal end 120b of the connector 120 and the stem portion 203 of the implant 200 are each provided with corresponding threads such that the distal end 120b of the connector 120 is fastened into the implant 200. In other words, the surgeon screws the distal end 120b of the connector 120 to the stem portion 203 of the implant 200 to couple the connector 120 and the implant 200.

At step 307, the impactor 160 is slidably coupled (or mounted) to the elongate member 140. In an exemplary embodiment, the impactor 160 is slid via the distal end 140b of the elongate member 140.

Additionally or optionally, depending on the bone density/quality at the implantation site, an appropriate weight of the impactor 160 (as shown in FIG. 1) is selected.

Alternatively, an impactor 160 having two or more sub units (for example, the first sub-unit 160a, and the second sub-unit 160b (as shown in FIGS. 6 and 7) may be selected to be slid over the elongate member 140.

At step 309, the elongate member 140 is coupled to the connector 120. In an exemplary embodiment, the elongate member 140 is coupled to the head portion 123 of the connector 120 by screwing the distal end 140b of the elongate member 140 within the cavity 123a of the connector 120.

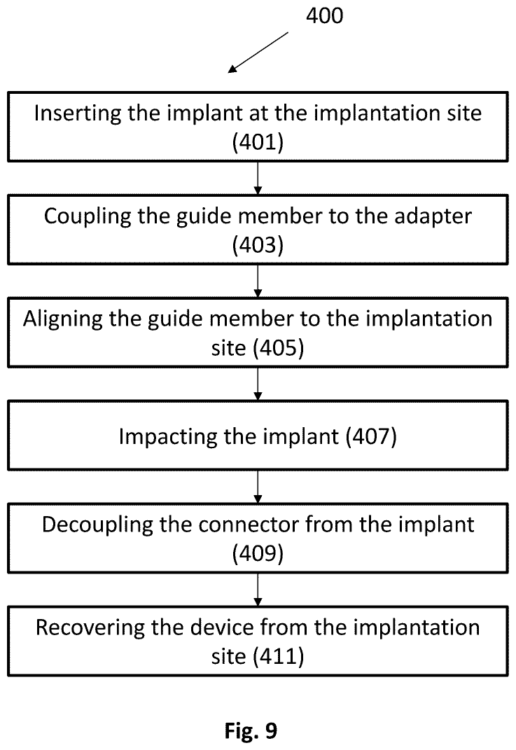

FIG. 9 depicts an exemplary embodiment of an implantation procedure 400 to insert the implant 200 at the implantation site using the device 100. For the implantation procedure 400, the device 100 is prepped by coupling the implant 200 as described above (with reference to FIG. 8).

The implantation procedure 400 commences at step 401 by inserting the implant 200 within the bone cavity at the implantation site. In an exemplary embodiment, the implant 200 is inserted by pushing the device 100 and/or implant 200 within the bone cavity until the implant 200 experiences resistance to the pushing of the implant 200.

At step 403, the guide member 130 is coupled to the adapter 110. In an exemplary embodiment, the axial projection 131 of the guide member 130 is removably coupled to one of the cavities 113a of the adapter 110. The coupling between the axial projection 131 of the guide member 130 and one of the cavities 113a of the adapter 110 enables the surgeon to set the pre-determined anteversion angle of the implant 200 (described in next step) for each patient as required.

At step 405, the surgeon aligns the guide member 130 of the device 100 perpendicular to the imaginary axis ‘x-x’ by rotating the device 100. Due to the projections 111c1 of the adapter 110, the implant 200 rotates along with the device 100. In an exemplary embodiment, the surgeon aligns the guide member 130 parallel to the long axis of the tibia while the tibia bone is flexed and held perpendicular to the femur bone. The said alignment of the guide member 130 sets the anteversion angle of the implant 200 with respect to the implantation site.

At step 407, the implant 200 is pushed inside the bone cavity at the implantation site by using the impactor 160 of the device 100. In an exemplary embodiment, the impactor 160 is slidably used, by the surgeon, to impact the head portion 123 of the connector 120, thereby transferring a pre-defined amount of force to the connector 120 from the impactor 160. The connector 120 transfers the pre-defined force to the implant 200, thereby enabling the implant 200 to be pushed within the bone cavity at the implantation site. The pre-defined amount of force is directly proportional to at least the weight of the impactor 160 and/or a distance between the stoppers 150 and the connector 120.

Additionally or optionally, if the impactor 160 having two or more sub units (for example, the first sub-unit 160a, and the second sub-unit 160b is used, the impactor 160 may be toggled between a first configuration and a second configuration as required by the surgeon. In the first configuration of the impactor 160, both the first and second sub units 160a, 160b are coupled to each other and configured to slide over the elongate member 140. In the first configuration of the impactor 160, the impactor 160 is used for sclerotic bones.

In the second configuration of the impactor 160, the second sub unit 160b is decoupled from the first sub unit 160a. Further, the first sub unit 160a is reversibly docked adjacent to the second stopper 150b. Thus, only the second sub unit 160b is configured to slide over the elongate member 140. In the second configuration of the impactor 160, the impactor 160 is used for osteoporotic bones.

At step 409, the connector 120 is decoupled from the implant 200. In an exemplary embodiment, the surgeon unscrews the distal end 120b of the connector 120 from the stem portion 203 of the implant 200 to decouple the connector 120 and the implant 200.

At step 411, the device 100 is recovered from the implantation site, thus leaving the implant 200 inside the bone cavity at the implantation site.

Although the implantation procedure 400 is described for impacting the implant 200 with the help of the impactor 160, the implant 200 may be impacted with the help of a mallet being hit on either the stoppers 150 or the head portion 123 of the connector 120. If the connector 120 is hit to impact the implant 200, the device 100 may be assembled, for example, without the elongate member 140, the stopper(s) 150 and/or the impactor(s) 160 and the same is within the scope of the teachings of the present disclosure.

Although the implantation procedure 400 is described to be performed by a surgeon, the implantation procedure 400 may be performed by an automatic entity, for example, a robotic arm (not shown) and the same is within the scope of the present disclosure.

The present disclosure will now be explained via the following examples.

Example 1 (Prior art): A stem prosthesis was inserted within a diseased femur bone to supplement the strength of the diseased bone. A mallet was used, by a surgeon, to hammer the stem prosthesis within the bone cavity of the femur bone. The alignment (i.e., the anteversion angle) of the prosthesis was roughly estimated by eyeballing the implantation site. During impaction of the implant within the bone cavity, the alignment of the implant was distorted. The prosthesis slipped and rotated during the impaction of the prosthesis. Further, due to use of mallet, the impaction force used to drive the prosthesis in the bone cavity was uneven (and varied from one surgeon to other) thereby causing the underlying bone to fracture.

Example 2 (Present disclosure): The device 100 of the present disclosure was assembled and used to insert the implant 200 within the bone cavity of the femur bone. The neck portion 201 of the implant 200 was oriented towards the medial side of the adapter 110 basis the first indicator 111c2 (and the second indicator) provided over the third section 111c of the tubular portion 111, and the third indicator 113b (and the fourth indicator 113c) provided over the flange portion 113. The stem portion 203 of the implant 200 was removably coupled with the adapter 110 by engaging the projections 111c1 of the adapter 110 with respective first recesses 203a of the stem portion 203. The said coupling between the adapter 110 and the implant 200 restricted rotation of the implant 200 with respect to the adapter 110 (for the entire duration of the implantation procedure). The distal end 120b of the connector 120 was fastened into the second recess 203b of the implant 200 to couple the connector 120 and the implant 200. The impactor 160 was slidably loaded over the elongate member 140. The distal end 140b of the elongate member 140 was fastened within the cavity 123a of the connector 120 to couple the elongate member 140 to the connector 120.

The implant 200 was inserted by pushing the device 100 and the implant 200 within the bone cavity until the implant 200 experienced resistance to the pushing of the implant 200. The axial projection 131 of the guide member 130 was removably coupled to one of the cavities 113a of the adapter 110. The surgeon aligned the guide member 130 parallel to the long axis of the tibia while the tibia bone was flexed and held perpendicular to the femur bone thereby setting the pre-determined anteversion angle of the implant 200 (for the entire duration of the implantation procedure). The impactor 160 was slidably used to impact the head portion 123 of the connector 120, thereby enabling the implant 200 to be pushed within the bone cavity at the implantation site using an even amount of force (which did not vary even after being impacted by different surgeons) for each impact of the impactor 160. After the implant 200 was completely inserted within the bone cavity of the femur bone, the surgeon disengaged the distal end 120b of the connector 120 from the stem portion 203 of the implant 200 to decouple the connector 120 and the implant 200. The device 100 was recovered from the implantation site, thus leaving the implant 200 inside the bone cavity of the femur bone.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim

1. A device (100), comprising:
an adapter (110) having a proximal end (110a), a distal end (110b) and a lumen (114) extending between the proximal end (110a) and the distal end (110b), including:
a tubular portion (111) including one or more projections (111c1) disposed towards the distal end (110b) of the adapter (110), the projections (111c1) removably couple the tubular portion (111) to an implant (200), the tubular portion (111) at least partially defining the lumen (114) of the adapter (110),
a flange portion (113) disposed coaxially to the tubular portion (111) at the proximal end (110a) of the adapter (110), the flange portion (113) at least partially defining the lumen (114) of the adapter (110), and
at least one set of a plurality of cavities (113a) disposed circumferentially on the flange portion (113), each cavity (113a) being at an offset with respect to the other cavity (113a), each cavity (113a) of the plurality of cavities (113a) corresponds to an anteversion angle being made by the implant (200) coupled to the tubular portion (111) of the adapter (110) with an implantation site;
a guide member (130) removably coupled to at least one of the cavities (113a) of the flange portion (113) of the adapter (110) to set the anteversion angle; and
a connector (120) having a proximal end (120a) and a distal end (120b), including:
a head portion (123), disposed at a proximal end (120a) of the connector (120); and
a shank portion (121) at least partially disposed within the lumen (114) of the adapter (110) through the flange portion (113), the distal end (120b) of the shank portion (121) is operationally coupled to the implant (200) for insertion within a bone cavity.

2. The device (100) as claimed in claim 1, the device (100) includes:
an elongate member (140) having a proximal end (140a) and a distal end (140b), the distal end (140b) of the elongate member (140) coupled to the head portion (123) of the connector (120);
one or more stoppers (150) coupled to the proximal end (140a) of the elongate member (140), and
at least one impactor (160) slidably disposed around the elongate member (140).

3. The device (100) as claimed in claim 1, wherein the tubular portion (111) and the flange portion (113) of the adapter (110) are either removably coupled or form an integral structure.

4. The device (100) as claimed in claim 1, wherein the shank portion (121) and the head portion (123) of the connector (120) are either removably coupled or form an integral structure.

5. The device (100) as claimed in claim 1, wherein a second engagement portion (121a) of the shank portion (121) is configured to transiently engage a first engagement portion (111a1) of the adapter (110).

6. The device (100) as claimed in claim 1, wherein the tubular portion (111) includes a second section (111b) provided with one or more cut outs (111b1) of a pre-defined shape and angular orientation enabling the connector (120) to freely rotate within the lumen (114) of the adapter (110) without letting the connector (120) slip out of the lumen (114) of the adapter (110).

7. The device (100) as claimed in claim 1, wherein the tubular portion (111) is provided with at least one first indicator (111c2) to correctly orient a medial side of the implant (200) with a medial side of the adapter (110).

8. The device (100) as claimed in claim 1, wherein the flange portion (113) is provided with at least one of a third indicator(s) (113b) indicating the medial side of the adapter (110) and a fourth indicator(s) (113c) indicating the lateral side of the adapter (110).

9. The device (100) as claimed in claim 1, wherein the anteversion angle corresponding to each of the cavities (113a) either increases or decreases from a medial side to a lateral side of the adapter (110).

10. The device (100) as claimed in claim 1, wherein the at least one set of a plurality of cavities (113a) include a first set of cavities (113a) and a second set of cavities (113a) disposed diametrically opposite to the first set of cavities (113a).

11. The device (100) as claimed in claim 1, wherein a second engagement portion (121a) of the shank portion (121) is disposed either proximal or distal to a first engagement portion (111a1) of the adapter (110).

12. The device (100) as claimed in claim 1, wherein the distal end (140b) of the elongate member (140) is coupled to a cavity (123a) of the head portion (123) of the connector (120).

13. The device (100) as claimed in claim 2, wherein the stoppers (150) include a first stopper (150a) disposed proximal to a second stopper (150b).

14. The device (100) as claimed in claim 2, wherein the impactor (160) includes:
a first sub-unit (160a);
a second sub unit (160b) removably coupled to the first sub-unit (160a); and
a docking means (161) for reversibly docking the first sub unit (160a) to the elongate member (140) adjacent to the stoppers (150).

15. An assembly comprising:
the device (100) as claimed in any of the claims above; and
an implant (200) having a neck portion (201) and a stem portion (203), the stem portion (203) having one or more first recesses (203a) and a second recess (203b), the first recesses (203a) of the stem portion (203) is removably coupled to corresponding projections (111c1) of the adapter (110), the second recess (203b) of the stem portion (203) is operationally coupled to the distal end (120b) of the connector (120).
